(19) United States
(12) Patent Application Publication
(10) Pub. No.: US 2025/0087308 A1
(43) Pub. Date: Mar. 13, 2025

(54) MACHINE LEARNING-BASED POLYMER SURFACE ENERGY PREDICTION SYSTEM

(71) Applicant: 3M INNOVATIVE PROPERTIES COMPANY, St. Paul, MN (US)

(72) Inventors: Richard B. Ross, Cottage Grove, MN (US); Shruti Venkatram, Atlanta, GA (US)

(21) Appl. No.: 18/580,774

(22) PCT Filed: Aug. 3, 2022
(86) PCT No.: PCT/IB2022/057222

§ 371 Date: Jan. 19, 2024

Related U.S. Application Data
(60) Provisional application No. 63/203,965, filed on Aug. 5, 2021.

(51) Int. Cl. G16C 20/30; G16C 20/70; G16C 60/00

(52) U.S. Cl. CPC G16C 20/30; G16C 20/70; G16C 60/00

(57) ABSTRACT

A method includes determining a plurality of molecular descriptors, each molecular descriptor of the plurality of molecular descriptors associated with at least one of an atomic scale property, a molecular scale property, and a compound-scale property. The method further includes selecting, by a selection operator and based on a minimization of an error, a subset of the plurality of molecular descriptors that affect a polymer surface energy, training a machine learning model to predict a polymer surface energy of a given polymer based on the subset of molecular descriptors and predicting, via the trained machine learning model, a surface energy of an input polymer.

### TECHNICAL FIELD

The disclosure relates to surface energy prediction.

### BACKGROUND

Consideration of surface energies provide an understanding of articles, materials, and products for many applications, such as adhesives, coatings, repellent materials, and applications involving wetting of substrates by materials. Current methods for determining polymer surface energies usually involve a trade-off between cost and accuracy. Experimental determinations are accurate but time-intensive while the converse is true for conventional computational methods.

### SUMMARY

In general, the disclosure describes systems and techniques for predicting or computationally determining surface energies of polymers. The systems and techniques disclosed may provide for computational prediction of polymer surface energies with improved accuracy and reduced cost.

In one example, this disclosure describes a method including determining a plurality of molecular descriptors, each molecular descriptor of the plurality of molecular descriptors associated with at least one of an atomic scale property, a molecular scale property, and a compound-scale property: selecting, by a selection operator and based on a minimization of an error, a subset of the plurality of molecular descriptors that affect a polymer surface energy: training a machine learning model to predict a polymer surface energy of a given polymer based on the subset of molecular descriptors; and predicting, via the trained machine learning model, a surface energy of an input polymer.

In another example, this disclosure describes a method including determining a plurality of molecular descriptors, each molecular descriptor of the plurality of molecular descriptors associated with at least one of an atomic scale property, a molecular scale property, and a compound-scale property: selecting, by a selection operator and based on a minimization of an error, a subset of the plurality of molecular descriptors that affect a polymer surface energy: predicting, via a trained machine learning model, a surface energy of an input polymer.

In another example, this disclosure describes a computer readable medium including instructions that when executed cause one or more processors to: determine a plurality of molecular descriptors, each molecular descriptor of the plurality of molecular descriptors associated with at least one of an atomic scale property, a molecular scale property, and a compound-scale property: select, by a selection operator and based on a minimization of an error, a subset of the plurality of molecular descriptors that affect a polymer surface energy: predict, via a trained machine learning model, a surface energy of an input polymer: and output the predicted surface energy.

In another example, this disclosure describes a means for determining plurality of molecular descriptors, each molecular descriptor of the plurality of molecular descriptors associated with at least one of an atomic scale property, a molecular scale property, or a compound-scale property: a means for selecting, based on a minimization of an error, a subset of the plurality of molecular descriptors that affect a polymer surface energy: a means for training a machine learning model to predict a polymer surface energy of a given polymer based on the subset of molecular descriptors: and a means for predicting, via the trained machine learning model, a surface energy of an input polymer.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a block diagram illustrating an example computing device configured to execute a polymer surface energy prediction technique, in accordance with the techniques of this disclosure.

FIG. 2A is a flowchart of an example method of training a machine informatics/machine learning (MI/ML) model for polymer surface energy prediction, in accordance with one or more techniques of this disclosure.

FIG. 2B is a flowchart of an example method of predicting a polymer surface energy via a trained MI/ML model, in accordance with one or more techniques of this disclosure.

FIG. 3 is a plot of an example dataset for training a material informatics machine learning (MI/ML) model, in accordance with the techniques described in this disclosure.

FIG. 4 is a plot of an example error of a MI/ML model as a function of training dataset size, in accordance with the techniques described in this disclosure.

FIG. 5 is a plot of an example comparison of the measured surface energy of each of a plurality of polymers versus the surface energy of each of the plurality of polymers predicted by an example MI/ML model, in accordance with the techniques described in this disclosure.

FIG. 6A is a plot of example measured polymer surface energy versus polymer surface energy predicted by a ML/MI model, in accordance with the techniques described in this disclosure.

FIG. 6B is a plot of example measured polymer surface energy versus polymer surface energy predicted by a first example conventional computation model.

FIG. 6C is a plot of example measured polymer surface energy versus polymer surface energy predicted by a second conventional computation model.

FIG. 7A is a plot of a plurality of example surface energy absolute differences between measured polymer surface energies and polymer surface energies predicted by a ML/MI model, in accordance with the techniques described in this disclosure.

FIG. 7B is a plot of a plurality of example surface energy absolute differences between measured polymer surface energies and polymer surface energies predicted by a first example conventional computation model.

FIG. 7C is a plot of a plurality of example surface energy absolute differences between measured polymer surface energies and polymer surface energies predicted by a second conventional computation model.

### DETAILED DESCRIPTION

In examples, the disclosure describes systems and techniques for predicting or computationally determining surface energies of polymers. In some examples, techniques and systems utilize a fingerprinting scheme for selecting a plurality of molecular descriptors that affect polymer surface energy, a selection operator to select a subset of the plurality of molecular descriptors based on minimization of an error value, and a material informatics machine learning (MI/ML) model trained to predict surface energies of a polymer based on the subset of molecular descriptors.

Rapid prediction of surface and interfacial energies with an MI/ML model streamlines the process to improve surface and interfacial energies for a variety of applications. Development and optimization of products, such as polymer products having particular surface and interfacial energies, may be slow due to the need to experimentally create the product and measure the surface and interfacial energies. Conventional computational prediction methods for polymer surface and interfacial energies suffer from poor prediction accuracy and offer only limited development improvements.

Surface energy (g) of solids, or surface tension of liquids, is generally defined as the work needed to create a surface at constant temperature, pressure, and composition. Surface energy is also related to interfacial energy (g₁₂) which is generally defined as the energy required to create an interface between two surfaces, such as an adhesive and a substrate.

Surface energy and interfacial energy are design properties of materials in many applications and in adhesion science, as well as in the development of polymeric coatings with specific wetting properties, oleophobicity, hydrophobicity, and the like. Surface and interfacial energies may also be design properties for polymer processing, e.g., for release of polymeric materials from molds.

As an example, surface and interfacial energies may be important in designing adhesives for bonding to low surface energy substrates, such as polypropylene, or high surface energy substrates, such as metals. A generally observed tendency is that low surface energy adhesives bond well to low surface energy substrates while high surface energy adhesives bond well to high surface energy substrates.

The work of adhesion (W_{adh}) between two surfaces can be calculated from the surface energy and the interfacial energy: W_{adh}=g₁+g₂−g₁₂. If W_{adh} is positive, the interface is stable and requires work to separate the two surfaces. If W_{adh} is negative, the interface is unstable and will separate spontaneously.

The interfacial energy between two surfaces can be calculated for non-polar or low-polar materials from the surface energies of the two materials via: g₁₂=(g₁^{0.5}−g₂^{0.5})². This equation does not work well for strongly polar or hydrogen bonding materials. For strongly polar or hydrogen bonding types of materials, an approach that has been shown to work well is to express the surface energy as a sum of dispersive and polar/hydrogen bonding terms, namely g=g_{d}+gₓ. The interfacial energy between two surfaces can then be calculated via g₁₂=(g_{d1}^{0.5}−g_{d2}^{0.5})²+(gₓ₁^{0.5}−gₓ₂^{0.5})².

Polar/hydrogen bonding surface energy components (gₓ) have been estimated by a number of methods and are available for many commonly used polymers. The polar/hydrogen bonding component may then be used to calculate the dispersive component from the overall surface energy of the material via g=g_{d}+gₓ.

If the surface energies of two materials to be bonded are measured experimentally or predicted by computational theoretical methodologies, the surface energies can then be used to calculate the interfacial energies from the equations above for many material interfaces. However, experimental measurement is high cost (e.g., in terms of labor, time, materials, etc.) and conventional computational methods are low accuracy, also resulting in high cost.

The systems and techniques disclosed herein provide for computational prediction of polymer surface energies with improved accuracy and reduced cost. In some examples, a method of implementing a MI/ML model to rapidly predict surface energies for polymers is disclosed. The predicted surface energies may then be used to calculate interfacial energies, via the equations above.

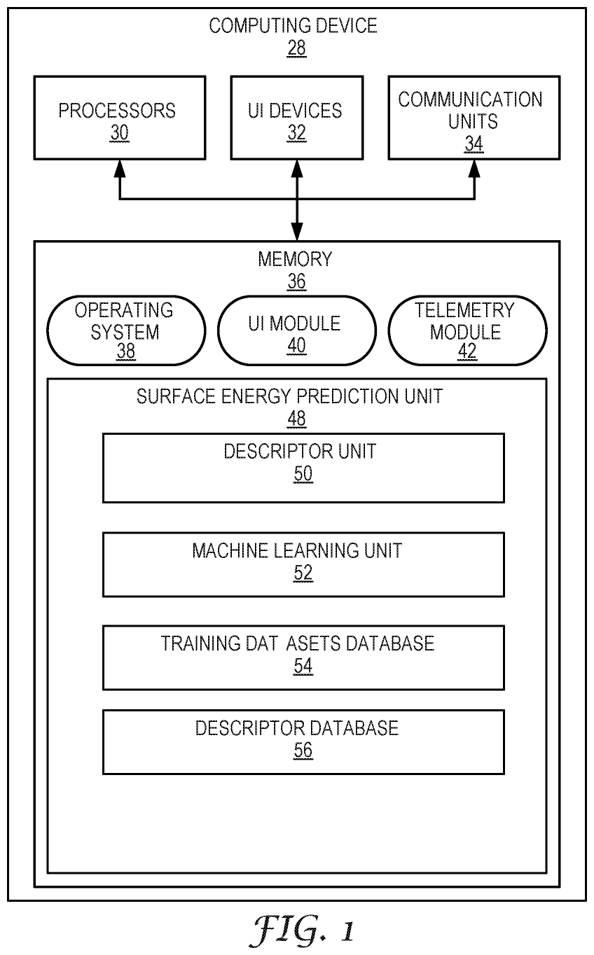

FIG. 1 is a block diagram illustrating an example computing device configured to execute a polymer surface energy prediction technique, in accordance with the techniques of this disclosure. The architecture of computing device 28 illustrated in FIG. 1 is shown for exemplary purposes only and computing device 28 is not limited to this architecture. In other examples. computing device 28 may be configured in a variety of ways. Computing device 28 may be configured to execute a MI/ML model-based surface energy prediction method. for example. by executing surface energy prediction unit 48.

As shown in the example of FIG. 1. computing device 28 includes one or more processors 30. one or more user interface (UI) devices 32. one or more communication units 34. and one or more memory units 36. Memory 36 of computing device 28 stores operating system 38. user interface (UI) module 40. telemetry module 42. and surface energy prediction unit 48. which are executable by processors 30. Each of the components. units or modules of computing device 28 are coupled (physically, communicatively, and/or operatively) using communication channels for inter-component communications. In some examples, the communication channels may include a system bus, a network connection, an inter-process communication data structure, or any other technology for communicating data.

Processors 30, in one example, may comprise one or more processors that are configured to implement functionality and/or process instructions for execution within computing device 28. For example, processors 30 may be capable of processing instructions stored by memory 36. Processors 30 may include, for example, microprocessors, a single-core processor, a multi-core processor. digital signal processors (DSPs). application specific integrated circuits (ASICs), field-programmable gate array (FPGAs), processing circuitry (e.g., fixed function circuitry. programmable circuitry, or any combination of fixed function circuitry and programmable circuitry) or equivalent discrete or integrated logic circuitry, or a combination of any of the foregoing devices or circuitry.

Memory 36 may be configured to store information (e.g., data and/or executable instructions) within computing device 28 during operation. Memory 36 may include a computer-readable storage medium or computer-readable storage device. In some examples, memory 36 may include one or more of a short-term memory or a long-term memory. Memory 36 may include, for example, one or more of random access memories (RAM). dynamic random access memories (DRAM), static random access memories (SRAM). on chip memory (e.g., in the case of SoC implementations). off chip memory, magnetic discs, optical discs, flash memories, or forms of electrically programmable memories (EPROM) or electrically erasable and programmable memories (EEPROM). In some examples, memory 36 is used to store program instructions for execution by processors 30. Memory 36 may be used by software or applications running on computing device 28 (e.g., surface energy prediction unit 48) to temporarily store information during program execution.

Computing device 28 may utilize communication units 34 to communicate with external devices via one or more networks and/or via wireless signals. Communication units 34 may be network interfaces, such as Ethernet interfaces, optical transceivers, radio frequency (RF) transceivers, or any other type of devices that can send and receive information. Other examples of interfaces may include Wi-Fi®, near field communication (NFC), or Bluetooth® radios. In some examples, computing device 28 utilizes communication units 34 to wirelessly communicate with an external device.

UI devices 32 may be configured to operate as both input devices and output devices. For example, UI devices 32 may be configured to receive tactile, audio, textual, or visual input from a user of computing device 28. In addition to receiving input from a user, UI devices 32 may be configured to provide output to a user or machine via tactile, audio, digital, or visual means. In one example, UI devices 32 may be configured to output content such as a graphical user interface (GUI) for display at a display device. UI devices 32 may include a presence-sensitive display (e.g., a so-called “touchscreen”) that displays a GUI and receives input from a user using capacitive, inductive, and/or optical detection at or near the presence-sensitive display.

Other examples of UI devices 32 include a mouse, a keyboard, a voice responsive system, video camera, microphone, or any other type of device for detecting a command from a user, or a sound card, a video graphics adapter card, or any other type of device for converting a signal into an appropriate form understandable to humans or machines. Additional examples of UI devices 32 include a speaker, a cathode ray tube (CRT) monitor, a liquid crystal display (LCD), organic light emitting diode (OLED),. or any other type of device that can generate or relay output data in a manner that is intelligible to a user or a machine.

Operating system 38 provides a multitasking operating environment for the operation of components of computing device 28. For example, operating system 38 facilitates the communication of UI module 40, telemetry module 42, and surface energy prediction unit 48 with processors 30, UI devices 32, communication units 34, and memory 36. UI module 40, telemetry module 42, and surface energy prediction unit 48 may each include program instructions and/or data stored in memory 36 that are executable by processors 30. For example, surface energy prediction unit 48 may include instructions that cause computing device 28 to perform one or more of the techniques described in this disclosure.

Computing device 28 may include additional components that, for clarity, are not shown in FIG. 1. For example, computing device 28 may include a battery to provide power to the components of computing device 28. Similarly, the components of computing device 28 shown in FIG. 1 may not be necessary in every example of computing device 28 that is consistent with this disclosure.

In the example illustrated in FIG. 1, surface energy prediction unit 48 includes descriptor unit 50, machine learning unit 52, training datasets database 54, and descriptor database 56. Descriptor unit 50 may comprise instructions for executing a hierarchical fingerprinting method to capture chemical and/or molecular descriptors that affect polymer surface energy. A molecular descriptor may be the final result of a logical and/or mathematical procedure which determines a numerical value and/or an experimental result based on chemical information encoded within a symbolic representation of a molecule. Descriptor unit 50 may be configured to execute feature engineering methods and/or processes to determine features and/or descriptors of a molecule, chemical, polymer, and the like, that are most relevant to surface energy. Descriptor unit 50 may provide structure and/or property information associated with the molecule, chemical, polymer, and the like. In some examples. descriptor unit 50 may be software, hardware, or a combination thereof configured to execute descriptor selection instructions, e.g., via execution of descriptor unit 50 by processors 30. Descriptor unit 50 may be configured to select a subset of molecular descriptors from a larger initial set of molecular descriptors relevant to polymers. In some examples, descriptor unit 50 may retrieve at least a portion of the larger initial set of molecular descriptors from molecular descriptor database 56. Molecular descriptor database 56 may be stored in memory 36 and/or retrieved from the memory of another device, such as a server, via communication units 34. In some examples, descriptor unit 50 may be configured to generate at least a portion of the larger initial set of molecular descriptors, e.g., via cheminformatics software such as RDKit (www.rdkit.org). Descriptor unit 50 may be configured to select the subset of molecular descriptors based on their effects on polymer surface energy.

For example, a user may select a plurality of polymers from which descriptor unit 50 may capture and/or select one or more molecular descriptors that affect polymer surface energy. The user may provide a polymer identifier, such as a polymer specification in the form of a line notation (e.g., an American Standard Code for Information Interchange (ASCII) character string) for describing the structure of a polymer that may be used with a simplified molecular-input line-entry system (SMILES). In some examples, the user may input the SMILE specification to computing device 28. In other examples the user may input a polymer identifier to computing device 28, and descriptor unit 50 may form the SMILE specification using the polymer identifier. Descriptor unit 50 may then retrieve one or more descriptors for each polymer, e.g., from a database, or generate one or more descriptors for each polymer, e.g., via cheminformatics software such as RDKit. Descriptor unit 50 may store the retrieved and/or generated descriptors as a large initial set of molecular descriptors, e.g., in descriptor database 56.

Descriptor unit 50 may be configured to categorize each molecular descriptor of the larger initial set of molecular descriptors as at least one of an atomic scale molecular descriptor, a molecular scale molecular descriptor, or a compound-scale molecular descriptor. For example. an atomic scale molecular descriptor may be a count of relevant atoms of a polymer, e.g., a count of halogen atoms, a count of oxygen atoms, a count of three-fold coordinated carbons, a count of four-fold coordinated carbons, and the like. Table 1 below includes a non-limiting list of relevant atoms. e.g., of a polymer and/or polymer fragment.

TABLE 1
Example polymer fragments including relevant atoms

| | | | |
|---|---|---|---|
| Aliphatic_COO | Nhpyrrole | furan | oxime |
| Aliphatic_OH | SH | guanido | para_hydroxylation |
| Aliphatic_OH_noTert | aldehyde | halogen | phenol |
| AromaticN | alkyl_carbamate | hdrzine | phenol_noOrthoHbond |
| Aromatic_COO | alkyl_halide | hdrzone | phos_acid |
| Aromatic_N | allylic_oxid | imidazole | phos_ester |
| Aromatic_NH | amide | imide | piperdine |
| Aromatic_OH | amidine | isocyan | piperzine |
| COO | aniline | isothiocyan | priamide |
| COO2 | aryl_methyl | ketone | prisulfonamd |
| C_O | azide | ketone_Topliss | pyridine |
| C_O_noCOO | azo | lactam | quatN |
| C_S | barbitur | lactone | sulfide |
| HOCCN | benzene | methoxy | sulfonamd |
| Imine | benzodiazepine | morpholine | sulfone |
| NH0 | bicyclic | nitrile | term_acetylene |
| NH1 | diazo | nitro | tetrazole |
| NH2 | dihydropyridine | nitro_arom | thiazole |
| N_O | epoxide | nitro_arom_nonor [symbol] | thiocyan |
| Ndealkylation1 | ester | nitroso | thiophene |
| Ndealkylation2 | ether | oxazole | unbrch_alkane |
| | | | urea |

[symbol]

indicates data missing or illegible when filed

A molecular scale molecular descriptor may comprise a population of pre-defined chemical building blocks, e.g., aldehydes, acids, aromatics, and the like. A compound-scale molecular descriptor may comprise quantitative structure-property relationship (QSPR) descriptors, e.g., van der Waals surface area, topological surface area, fraction of rotatable bonds, and the like. In some examples, descriptor unit 50 may be configured to categorize molecular descriptors hierarchically based on length scale. In some examples, descriptor unit 50 may be configured to categorize hundreds of molecular descriptors from the larger initial set of molecular descriptors. In some examples, descriptor unit 50 may be configured to categorize molecular descriptors by the length scale of a polymer or polymers. In some examples, descriptor unit 50 may be configured to categorize and/or classify ID molecular descriptors across the length scale of one or more polymers.

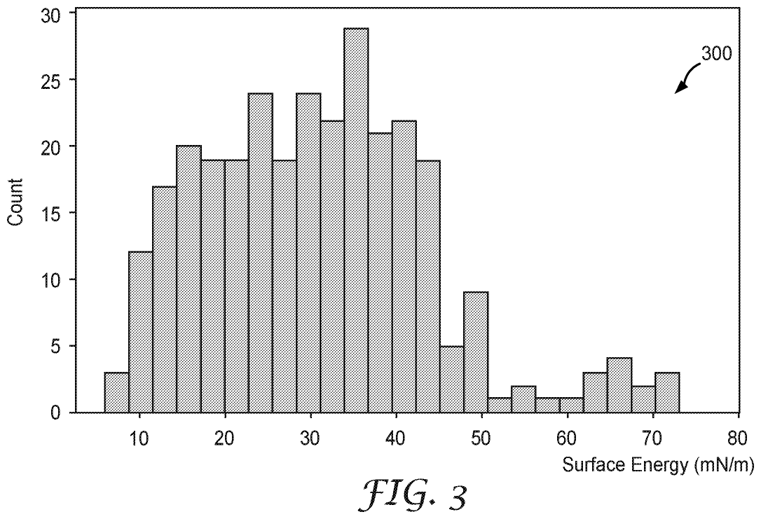

Descriptor unit 50 may be configured to select a subset of molecular descriptors based on each descriptor's effects on polymer surface energy from the larger, categorized set of molecular descriptors. For example, descriptor unit 50 may be configured to select the subset of molecular descriptors by executing regression methods including shrinkage (e.g., coefficient elimination) to select descriptors with the greatest effects on polymer surface energy. One example of such a technique that descriptor nit 50 may use is a least absolute shrinkage and selection operator (LASSO) that minimizes the sum of squared errors (between calculated and experimentally determined surface energies) with a bound on the sum of the absolute values of the LASSO coefficients. in this case. the descriptors. In the example of FIG. 3 below. descriptor unit 50 is configured to categorize a set of 150 descriptors from a larger initial set of molecular descriptors relating to a training dataset of 301 chemically diverse polymers (e.g., from training datasets database 54 described below) and select a subset of descriptors (e.g., 30 descriptors. although greater or fewer numbers of descriptors may be used in examples consistent with this disclosure) relevant to polymer surface energy via executing LASSO.

In some examples, a user may select and/or deselect one or more descriptors of the larger initial set of descriptors. For example, a user may know that one or more descriptors of the larger initial set of descriptors have an effect on polymer surface energy, and the user may select those one or more descriptors for inclusion with the subset of descriptors before and/or without either of categorization or selection (e.g., via regression methods) by descriptor unit 50. In other examples. a user may know that one or more descriptor of the large initial set of descriptors has little or no effect on polymer surface energy and a user may omit those one or more descriptors from the subset of descriptors before and/or without either of categorization or selection by descriptor unit 50, or may deselect those one or more descriptors if selected by descriptor unit 50. In other words, a user may modify the larger initial set of descriptors retrieved and/or generated, and/or may modify the selected subset of descriptors.

Training datasets database 54 may include a plurality of polymers and the respective surface energies or each of the plurality of polymers. The surface energies may be known. measured. or otherwise reliably determined. Training datasets database 54 may further include details regarding the polymers, such as molecular composition, class, type, and/or categorization. and physical, electrical, and/or chemical properties. In some examples. training datasets database 54 may be curated from a chemical or polymer database. built via experimentally making polymers and measuring their physical, electrical. and/or chemical properties and inputting the measured properties and any other polymer information (e.g., composition, structure, experimental or formulation details. and the like) by a user to training datasets database 54, or both. In some examples, training datasets database 54 may include the example dataset of FIG. 3 described below. For example, training datasets database 54 may include 301 chemically diverse polymers, each of which may be numerically presented via one or more relevant molecular descriptors. In other words, each polymer may be associated with one or more molecular descriptors retrieved and/or generated as described above, each descriptor comprising a numerical value representing an aspect of the polymer, e.g., a chemical composition, a structure, a property, and the like.

Descriptor database 56 may include a plurality of molecular descriptors suitable for predicting the surface energies of polymers. For example, descriptor database 56 may include any of the larger initial set of molecular descriptors, a set of descriptors categorized by descriptor unit 50 into atomic, molecular, and/or compound-scale molecular descriptors, and/or a selected subset of molecular descriptors, e.g., selected via descriptor unit 50 as described above. Descriptor database 56 may include a plurality of molecular descriptors retrieved and/or generated and selected by descriptor unit 50 based on one or more training datasets of training datasets database 54. Descriptor database 56 may include updated descriptors and/or new descriptors added. e.g., via machine learning unit 52.

Machine learning unit 52 may be configured to predict a surface energy of a polymer based on one or more descriptors, e.g., from descriptor database 56. In some examples. machine learning unit 52 may be a single-fidelity machine learning model. In some examples, machine learning unit 52 may implement Gaussian Process Regression (GPR) with a radial basis function (RBF) kernel configured to map one or more polymers to respective surface energies. GPR uses a Bayesian framework in which a Gaussian process is used to obtain the mapping from a polymer to its surface energy based on an available training dataset of polymers and associated descriptors and a Bayesian prior, which is incorporated using the kernel function. In some examples, machine learning unit 52 may implement GPR with cross-validation, e.g., GPR comprising a five-fold cross-validation.

The techniques, devices, and systems disclosed herein may provide an improvement in accuracy at a reduced cost, e.g., relative to conventional computational methods of predicting, and/or experimental methods of determining, polymer surface energy. For example, descriptor unit 50 and machine learning unit 52 may be configured to provide computational prediction according to methods with a greater emphasis and/or focus on polymer attributes relevant to polymer surface energy. By way of contrast, conventional computational methods for polymer surface energy prediction may approximate surface energy property/structure relationships by correlating a relatively small number (e.g., one, two, five, ten) of inherent properties to semi-empirical equations by fitting to experimental data, or approximate group contribution surface energy property/structure relationships by correlating chemical groups to experimental data, which requires a database of chemical fragments with group contributions to properties. The techniques, devices, and systems disclosed herein are predictive based on polymer composition and do not require a database of chemical group contributions. The techniques, devices, and systems disclosed may provide fast (e.g., within hours, within minutes, or within seconds) or potentially even substantially instantaneous polymer surface energy prediction with improved accuracy.

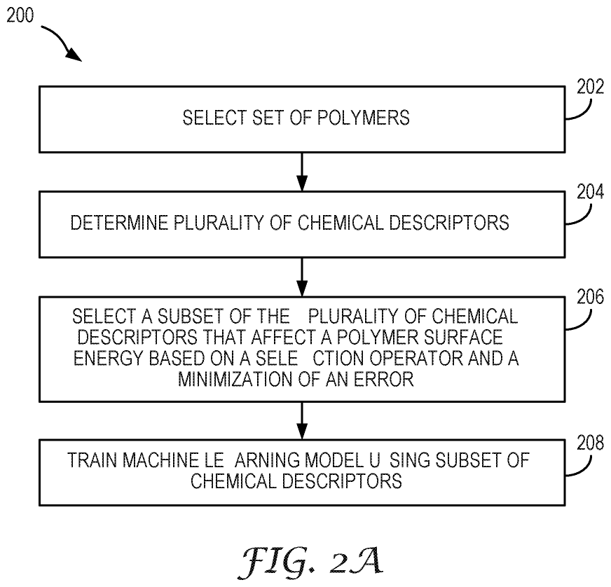

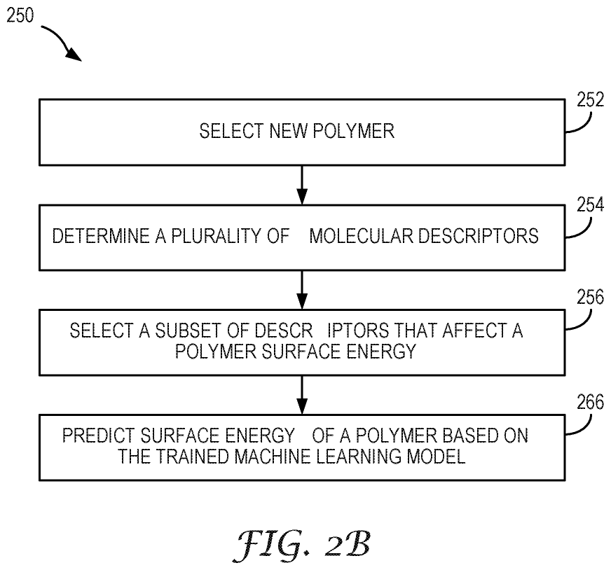

FIG. 2A is a flowchart of an example method 200 of training a machine informatics/machine learning (MI/ML) model for polymer surface energy prediction. in accordance with one or more techniques of this disclosure. Although method 200 is discussed using computing device 28 of FIG. 1, it is to be understood that the methods discussed herein may include and/or utilize other systems and methods in other examples.

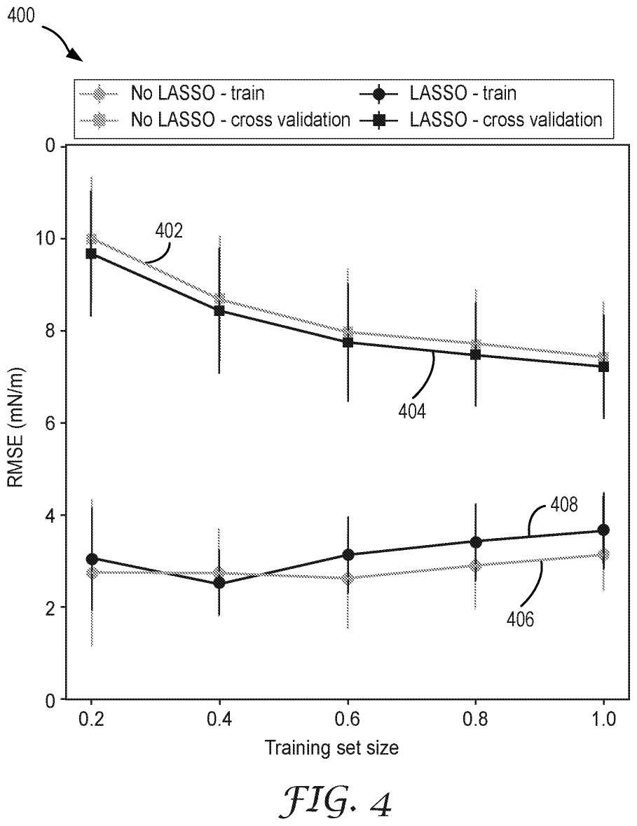

Computing device 28 may select a set of polymers and/or polymer compositions (202). For example, computing device 28 may receive a selection input via one or more of UI devices 32. For example, a user may select a population of chemically diverse polymers having a range of polymer surface energies. In the examples of FIGS. 4-8, 301 chemically diverse polymers are selected.

Computing device 28 may determine a plurality of molecular descriptors that control a polymer surface energy (204). Computing device 28 may retrieve, receive, and/or generate a plurality and/or a set of molecular descriptors, e.g., a large initial set of molecular descriptors. For example, computing device 28 may generate a large initial set of molecular descriptors via cheminformatics software and based on a plurality of polymers and their respective surface energies. In other examples, computing device 28 may retrieve and/or receive the larger initial set of molecular descriptors from a database, such as descriptor database 56. Computing device 28 may categorize each molecular descriptor of the larger initial set of molecular descriptors as at least one of an atomic scale molecular descriptor, a molecular scale molecular descriptor, or a compound-scale molecular descriptor. In some examples, the plurality of molecular descriptors affect a homopolymer surface energy.

Computing device 28 may categorize a polymer according to an atomic scale property including a count of relevant atoms, the relevant atoms including at least one of a halogen, oxygen, a three-fold coordinated carbon, or a four-fold coordinated carbon. Computing device 28 may categorize a polymer according to a molecular scale property including a count of functional groups, the functional groups including a count of at least one of an aldehyde group, an acid group, or an aromatic group. Computing device 28 may categorize a polymer according to a compound scale property including at least one of a van der Waals surface area, a topological surface area, or a fraction of rotatable bonds.

Computing device 28 may select a subset of molecular descriptors that affect a polymer surface energy based on a minimization of an error (206). For example, computing device 28 may execute a hierarchical fingerprinting method to capture chemical and/or molecular descriptors that affect polymer surface energy. In some examples, computing device 28 may select a subset of molecular descriptors based on each descriptor's effects on polymer surface energy from the larger, categorized set of molecular descriptors. In some examples, computing device 28 may execute a LASSO to select the subset of molecular descriptors.

In some examples, computing device 28 may process user input data received via one or more of UI devices 32 to select and/or deselect one or more descriptors of the larger initial set of descriptors. For example, one or more descriptors of the large initial set of descriptors may be known to have an effect on polymer surface energy, and a user may select those one or more descriptors for inclusion within the subset of descriptors before and/or without either of categorization or selection (e.g., via regression methods) at blocks (204) and/or (206). In other examples, one or more descriptors of the larger initial set of descriptors may be known to have little or no effect on polymer surface energy, and a user may omit those one or more descriptor from the subset of descriptors before and/or without either of categorization or selection by computing device 28, or may deselect those one or more descriptors if previously selected by computing device 28. In other words, a user may modify the large initial set of descriptors retrieved and/or generated, and/or may modify the selected subset of descriptors.

Computing device 28 may train a machine learning (ML) model to predict a polymer surface energy of a given polymer based on the subset of molecular descriptors (208). For example, computing device 28 may train a GPR ML model with an RBF kernel to map one or more polymers to respective surface energies using a training dataset of training datasets database 54. In some examples, the one or more training polymers may be homopolymers. In examples describe below, a dataset includes 301 distinct and chemically diverse polymers having known or determined surface energies ranging from 7 mN/m to 72 mN/m (e.g., as illustrated in FIG. 3). Computing device 28 may use 241 of the polymers of the dataset to train the GPR ML model, and reserve 60 of the polymers for testing and/or validation of the trained GPR ML model, e.g., five-fold cross-validation.

Computing device 28 may train a ML model with a training dataset comprising a plurality of polymers. each polymer including a plurality of polymer classes. For example, computing device 28 may train the ML model with a training dataset including at least one of a polyoxide, a polyvinyl, a polyolefin, a polyamide, or a polyether. Computing device 28 may train a ML model with a training dataset that further includes a plurality of chemical moieties including at least one of hydrogen, carbon, nitrogen, oxygen, sulfur, silicon, fluorine, chlorine, or bromine. In some examples, computing device 28 may train the ML model with a training dataset including at least one experimentally determined surface energy of at least one of the plurality of polymers of the training dataset.

FIG. 2B is a flowchart of an example method 250 of predicting a polymer surface energy via a trained MI/ML model, in accordance with one or more techniques of this disclosure. Although method 250 is discussed using computing device 28 of FIG. 1, it is to be understood that the methods discussed herein may include and/or utilize other systems and methods in other examples.

Computing device 28 may select a polymer and/or polymer composition (252). For example, computing device 28 may generate, retrieve, and/or receive. e.g., from a user or another computing device, a new polymer, and/or a specification of a new polymer, for surface energy prediction, and may provide information relating to the new polymer to computing device 28.

Computing device 28 may determine a plurality of molecular descriptors (254). For example, computing device 28 may retrieve, receive, and/or generate a plurality and/or a set of molecular descriptors, e.g., a large initial set of molecular descriptors, as described above with respect to FIG. 2A. for the user-selected polymer and/or polymer composition.

Computing device 28 may select a subset of descriptors that affect a polymer surface energy (256). In some examples, computing device 28 may select one or more of the same descriptors as the subset of molecular descriptors determined at block (206) of method 200 of FIG. 2A. For example, if computing device 28 determines molecular descriptors that includes all of the subset of molecular descriptors that affect polymer surface energy selected at block (206) of method 200 of FIG. 2A, computing device 28 might select the same subset of descriptors relating to the new polymer and/or polymer composition, e.g., the same subset of descriptors but having descriptor values relating to the new polymer and/or polymer composition. If computing device 28 determines molecular descriptors that do not include all of the subset of molecular descriptors that affect polymer surface energy selected at block (206) of method 200 of FIG. 2A. e.g., if there is not enough information regarding a new polymer to retrieve and/or generate all of the subset of molecular descriptors, computing device 28 may select at least a portion of the same descriptors as the subset of molecular descriptors selected at block (206) of method 200 of FIG. 2A.

Computing device 28 may predict a surface energy of an input polymer via the trained machine learning model (266). For example, computing device 28 may execute the trained GPR ML model to predict the surface energy of a polymer. In the examples described in FIGS. 3-6 below, computing device 28 may predict the surface energies of the 60 reserved polymers. e.g., to validate and/or test the performance of the trained GPR ML model. In some examples, the input polymer may be a homopolymer. In some examples, computing device 28 may predict a surface energy of an input polymer using a machine learning model without group contribution surface energy property/structure relation methods. without group additive contribution methods, and/or without a database of chemical group contributions. In some examples, computing device 28 may execute the trained GPR ML model with cross-validation. e.g., a five-fold cross-validation.

In some examples, training the machine learning model at block (208) of the method 200 of FIG. 2A may be optional. For example, computing device 28 may predict a surface energy of an input polymer without training the machine learning model. In some examples, the machine learning model may have been previously trained. may not require training, or may be trained “on the fly” and over time via predicting a plurality of input polymers and receiving information related to the surface energy and/or accuracy of the predictions of the surface energies of at least a portion of the input polymers.

FIG. 3 is a histogram 300 of an example dataset for training and validation of a material informatics machine learning (MI/ML) model, in accordance with the techniques described in this disclosure. In the example shown, the dataset includes 301 distinct polymers. The surface energies of each of the 301 polymers are experimentally determined and/or measured. The dataset of the example is curated from a database from National Institute for Materials Science (NIMS) of Tsukuba, Japan, https://mits.nims.go.jp/en/. The dataset comprises several polymer classes including polyoxides, polyvinyls, polyolefins, polyamides, and polyethers and includes a plurality of diverse chemical moieties that include at least one of hydrogen, carbon, nitrogen, oxygen, sulfur, silicon, fluorine, chlorine, or bromine.

FIG. 4 is a set of four plots 402-408 illustrating example root-mean squared errors of a MI/ML model as a function of training dataset size. in accordance with the techniques described in this disclosure. Plots 402-208 represent learning curves of the MI/ML model (e.g., a GPR ML model with a RBF kernel). Plots 402-408 illustrate the average training and cross-validation root-mean-squared-error (RMSE) of the ML/MI model as a function of training set size. e.g., as a decimal percentage from 0.0-1.0 where 1.0 is 100%. of the 241 polymers. The validation set of FIG. 4 refers to fractions of the 241 polymer training set as shown on the horizontal (x-) axis, all of which are distinct from the 60 polymers used for ML/MI model testing. The error bars represent 1 standard deviation of the average RMSE values over 20 iterations of the ML/MI model. In the example shown, plots 402 and 406 are learning curves of the ML/MI model trained using a set of descriptors selected according to the fingerprinting method described above, e.g., categorized via atomic, molecular, and compound-scale descriptors but without reduction via LASSO. Plots 404 and 408 are learning curves of the ML/MI model trained using a set of descriptors selected according to the fingerprinting method described above, e.g., with reduction of the descriptors used to via LASSO to a subset of the categorized set of descriptors.

In the example shown, the cross-validation RMSE of the ML/MI model trained with all initial descriptors (e.g., plots 402 and 406) and LASSO reduced descriptors (e.g., plots 404 and 408) decrease with increasing training set size. The ML/MI model trained with LASSO-reduced descriptors, on average, have lower test RMSE than the ML/MI model trained without reducing the set of descriptors via LASSO. By way of these examples. LASSO regression is shown to be an effective method for eliminating irrelevant and/or less relevant descriptors, thereby simplifying and reducing the time and/or computing power consumed by the ML/MI model. For example, LASSO may identify descriptors pertinent to the surface energy of a polymer, such as polar chemical moieties like halogens, functional groups like nitrile, carbonyl, amide, and amine. LASSO may also identify large-scale descriptors like the total polar surface area (TPSA) and log P of a polymer as important and/or relevant descriptors for determining surface energy.

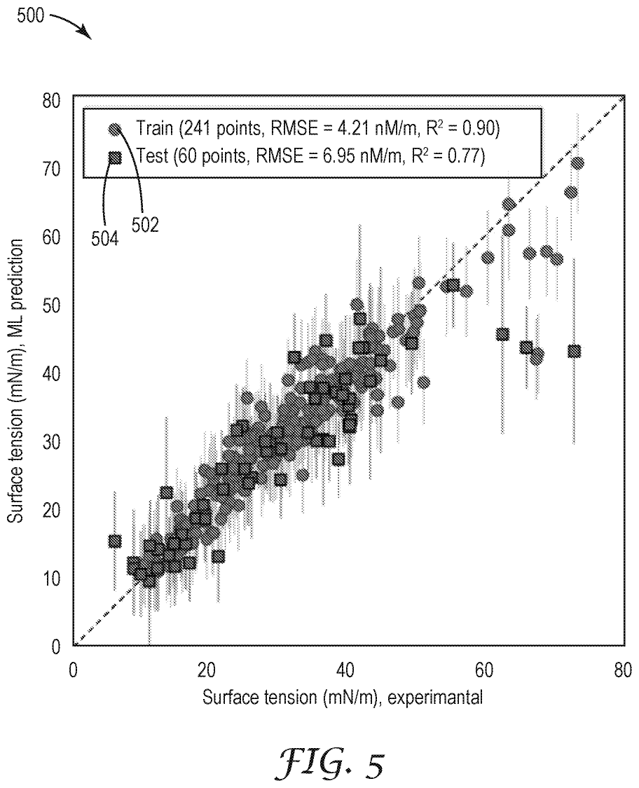

FIG. 5 is a plot 500 of an example comparison of the measured surface energy of each of a plurality of polymers of the dataset of FIG. 3 versus the surface energy of each of the plurality of polymers predicted by an example MI/ML model in accordance with the techniques described in this disclosure. In the example shown, data points 502 correspond to the 241 training polymers, and data points 504 correspond to the 60 test polymers.

In the example shown, an ML/MI model trained on the training set of 241 polymers predicted. e.g., according to method 200 described above, the surface energies of the 241 training polymers with an average RMSE of 4.21 mN/m and results in a linear correlation R-squared value of 0.90 with respect to the experimentally measured surface energies of the 241 training polymers. The ML/MI model predicted the surface energies of the test polymers with an average RMSE of 6.95 mN/m and a linear correlation R-squared value is 0.77 with respect to the experimentally measured surface energies of the 60 reserved test polymers. In the example shown, the error bars illustrate the ML/MI model (e.g., the GPR) uncertainty. In the example shown, predictions for polymers with higher surface energies, e.g., greater than 60 mN/m. are less accurate due to underrepresentation of higher surface energy polymers in the training data set, e.g., as illustrated in FIG. 3. In some examples. including a greater number of training polymers to the training data set may increase the prediction accuracy of the ML/MI model.

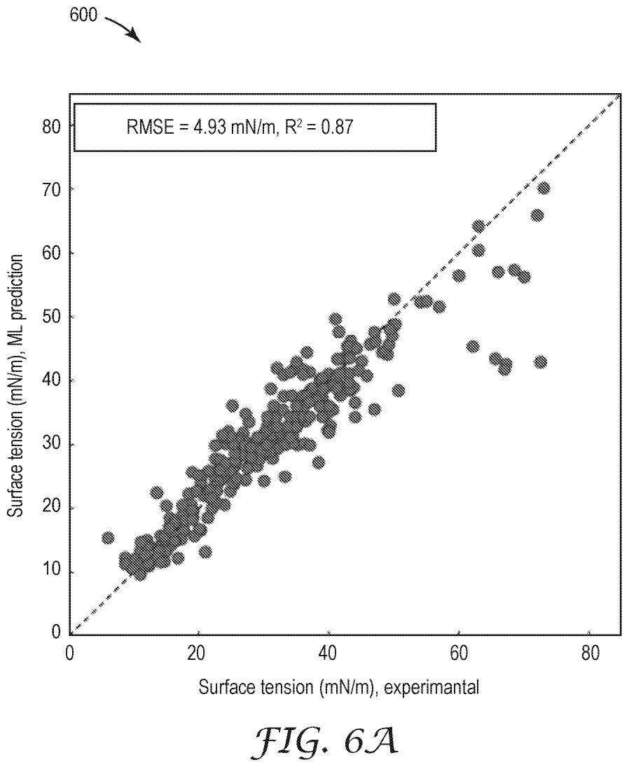

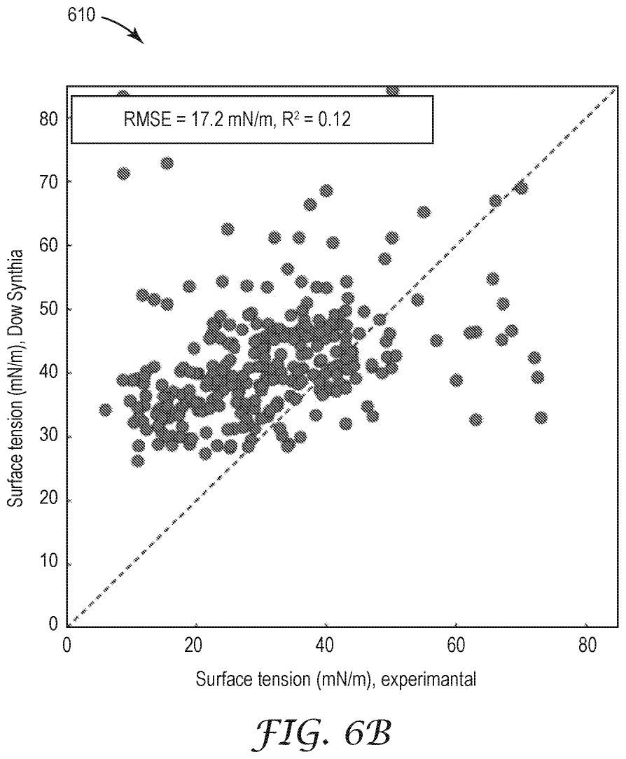

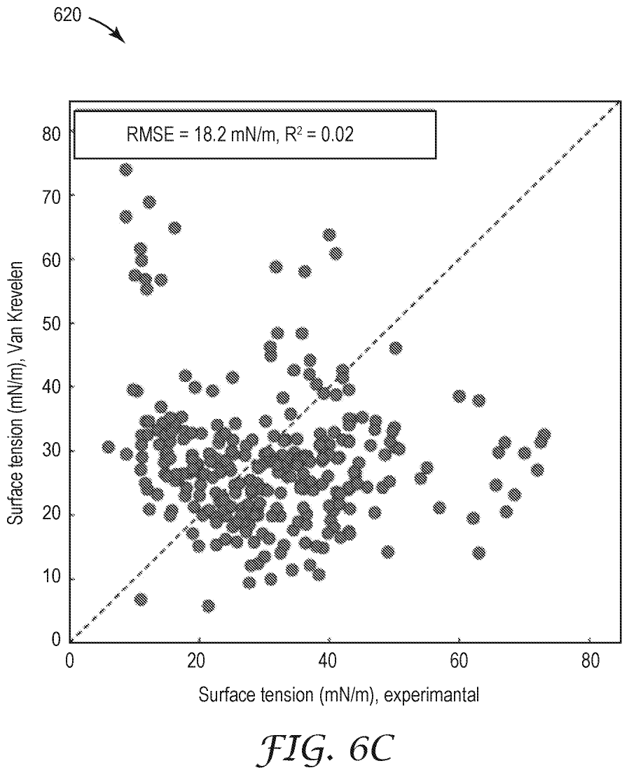

FIGS. 6A-6C are plots of an example measured surface energy of each of a plurality of polymers versus the surface energy of each of the plurality of polymers predicted by an example computational model and illustrate a comparison of three different polymer surface energy computation models. FIG. 6A is a plot 600 of measured polymer surface energy versus polymer surface energy predicted by a ML/MI model. in accordance with the techniques described in this disclosure. FIG. 6B is a plot 610 of measured polymer surface energy versus polymer surface energy predicted by a first example conventional computation model. and FIG. 6C is a plot 620 of measured polymer surface energy versus polymer surface energy predicted by a second conventional computation model. The measured versus predicted surface energies are of all 301 polymers of the example dataset described above.

In the example shown in FIG. 6B. the first example conventional computation model is a Dow Synthia model for predicting surface energies uses the polymer property/structure prediction methodology developed by J. Bicerano of Dow Chemical as implemented in the program Synthia from Dassault Systemes/Biovia. https://www.3ds.com/fileadmin/PRODUCTS-SERVICES/BIOVIA/PDF/BIOVIA-Material-Studio-synthia.pdf. In the example shown in FIG. 6C. the second example conventional computation model is a Van Krevelen model for predicting surface energies uses the polymer properties group additive predictive method developed by D.W. van Krevelen as implemented in the program Molecular Modeling Plus from Norgwyn Montgomery Software, http://www.norgwyn.com/mmpplus.html.

In the example of FIG. 6A, which illustrates the performance of a ML/MI model trained according to aspects of this disclosure, the ML/MI model average prediction RMSE is 4.93 mN/m and linear correlation R-squared value is 0.90 with respect to the experimentally measured polymer surface energies. The ML/MI model in the examples shown provides a greater prediction accuracy than the Dow Synthia and Van Krevelen predictions, with average RMSEs of 17.2 mN/m and 18.2 mN/m and linear correlation R-squared values of 0.12 and.02. respectively.

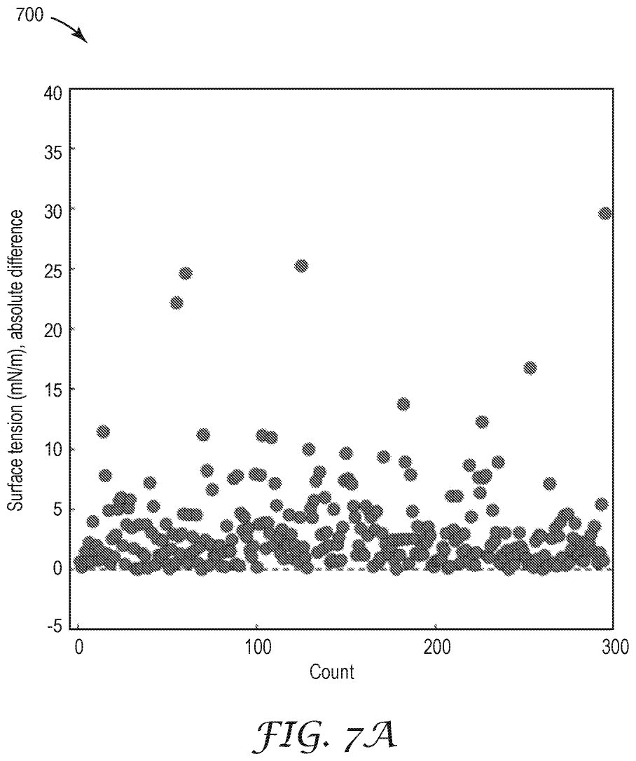

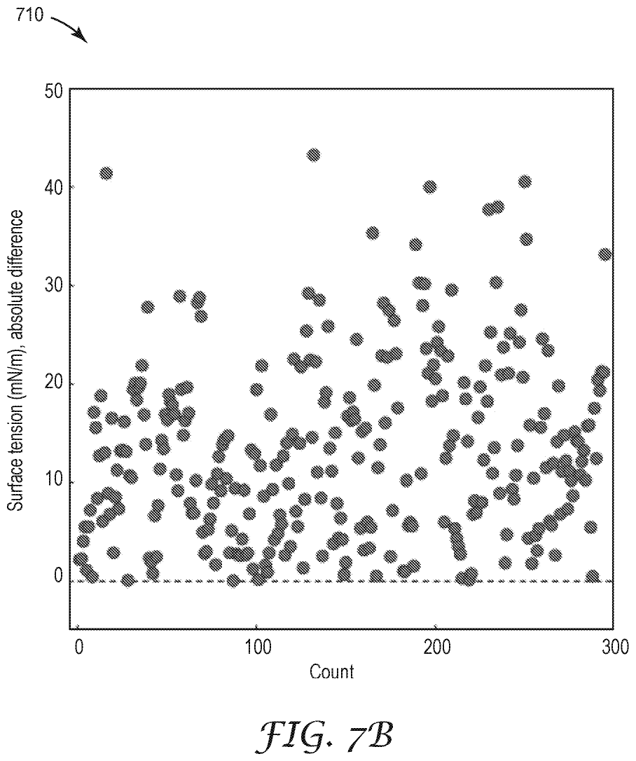

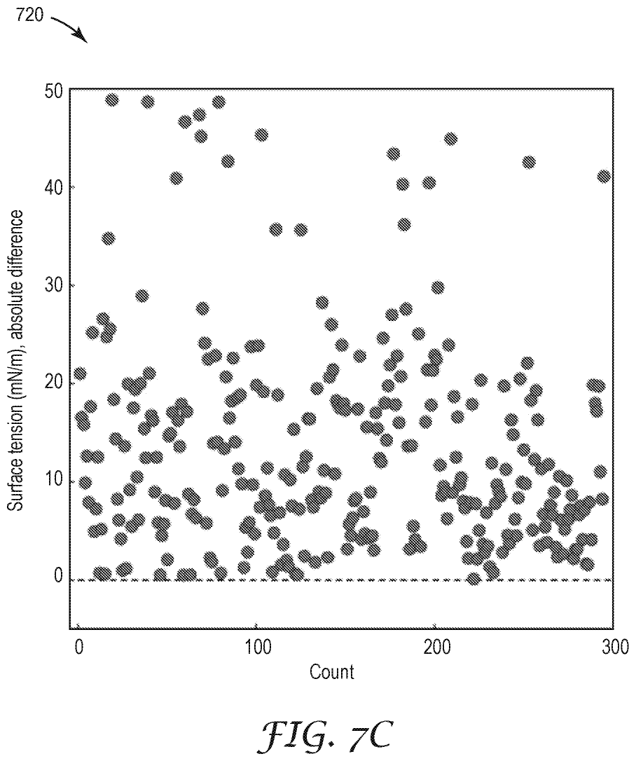

FIGS. 7A-7C are plots of a plurality of example surface energy absolute differences (e.g., difference magnitude) between measured polymer surface energies and predicted polymer surface energies predicted by the three different polymer surface energy computation models of FIGS. 6A-6C. FIG. 7A is a plot 700 of a plurality of example surface energy absolute differences between measured polymer surface energies and polymer surface energies predicted by a ML/MI model, in accordance with the techniques described in this disclosure. FIG. 7B is a plot 710 of a plurality of example surface energy absolute differences between measured polymer surface energies and polymer surface energies predicted by a first example conventional computation model, and FIG. 7C is a plot 720 of a plurality of example surface energy absolute differences between measured polymer surface energies and polymer surface energies predicted by a second conventional computation model. Plots 7A-7C illustrate the same measured/predicted polymer surface energies as plots 6A-6C. but plotted as a magnitude of the difference between the measured and predicted values as a function of polymer (e.g., the x-axis) rather than the measured vs. predicted plots of FIGS. 6A-6C.

The examples shown FIGS. 7A-7C visually illustrate the accuracy of each computation model for the entire data set of polymers, e.g., all 301 polymers of the example dataset described above. A perfect match between prediction and measurement would have all data points along the x-axis with an absolute difference of zero. The larger the distance of the datapoints from the x-axis. such as with FIGS. 7B-7C. the less accurate the predictions given by the model.

As shown in FIG. 7A is, the accuracy of the predictions of the ML/MI model is greater than the conventional computation models of FIGS. 7B and 7C. e.g., the absolute differences between the measure polymer surface energies and the polymer surface energies predicted by the ML/MI model are less than the absolute differences between the measured polymer surface energies and the polymer surface energies predicted by either conventional model of FIGS. 7B-7C. FIG. 7A also illustrates that the absolute differences between measured and ML/MI model predicted polymer surface energies are clustered around low. e.g., 5 mN/m or less, absolute differences much more than the absolute differences between measured and conventional computation model predicted polymer surface energies as shown in FIGS. 7B-7C.

The techniques described in this disclosure may be implemented, at least in part, in hardware, software, firmware, or any combination thereof. For example, various aspects of the described techniques may be implemented within one or more processors. including one or more microprocessors, digital signal processors (DSPs), application specific integrated circuits (ASICs), field programmable gate arrays (FPGAs), processing circuitry (e.g., fixed function circuitry, programmable circuitry, or any combination of fixed function circuitry and programmable circuitry), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware, firmware, or software components. Rather, functionality associated with one or more modules or units may be performed by separate hardware, firmware, or software components. or integrated within common or separate hardware. firmware, or software components.

The techniques described in this disclosure may also be embodied or encoded in an article of manufacture including a computer-readable storage medium encoded with instructions. Instructions embedded or encoded in an article of manufacture including a computer-readable storage medium, may cause one or more programmable processors, or other processors, to implement one or more of the techniques described herein, such as when instructions included or encoded in the computer-readable storage medium are executed by the one or more processors. Computer readable storage media may include random access memory (RAM). read only memory (ROM). programmable read only memory (PROM). erasable programmable read only memory (EPROM). electronically erasable programmable read only memory (EEPROM), flash memory. a hard disk. a compact disc ROM (CD-ROM). a floppy disk. a cassette, magnetic media, optical media, or other computer readable media. In some examples, an article of manufacture may include one or more computer-readable storage media.

In some examples, a computer-readable storage medium may include a non-transitory medium. The term “non-transitory” may indicate that the storage medium is not embodied in a carrier wave or a propagated signal. In certain examples. a non-transitory storage medium may store data that can, over time, change (e.g., in RAM or cache).

The following examples may illustrate one or more aspects of the disclosure:

Example 1: A method includes determining a plurality of molecular descriptors, each molecular descriptor of the plurality of molecular descriptors associated with at least one of an atomic scale property, a molecular scale property, and a compound-scale property: selecting, by a selection operator and based on a minimization of an error, a subset of the plurality of molecular descriptors that affect a polymer surface energy: training a machine learning model to predict a polymer surface energy of a given polymer based on the subset of molecular descriptors: and predicting, via the trained machine learning model, a surface energy of an input polymer.

Example 2: The method of example 1, wherein the polymer is a homopolymer.

Example 3: The method of any of examples 1 and 2, wherein the atomic scale property comprises a count of relevant atoms.

Example 4: The method of example 3, wherein the relevant atoms comprise at least one of a halogen, oxygen, a three-fold coordinated carbon, or a four-fold coordinated carbon.

Example 5: The method of any of examples 1 through 4, wherein the molecular scale property comprises a count of functional groups.

Example 6: The method of example 5, wherein the count of functional groups comprises a count of at least one of an aldehyde group, an acid group, or an aromatic group.

Example 7: The method of any of examples 1 through 6, wherein the compound scale property comprises at least one of a van der Waals surface area, a topological surface area, or a fraction of rotatable bonds.

Example 8: The method of any of examples 1 through 7, wherein the selection operator comprises a least absolute shrinkage and selection operator (LASSO).

Example 9: The method of any of examples 1 through 8, wherein the machine learning model comprises a Gaussian Process Regression (GPR) comprising a radial basis function (RBF) kernel.

Example 10: The method of example 9, wherein the GPR further comprises a five-fold cross-validation.

Example 11: A method includes determining a plurality of molecular descriptors, each molecular descriptor of the plurality of molecular descriptors associated with at least one of an atomic scale property, a molecular scale property, and a compound-scale property: selecting, by a selection operator and based on a minimization of an error, a subset of the plurality of molecular descriptors that affect a polymer surface energy: predicting, via a trained machine learning model, a surface energy of an input polymer.

Example 12: The method of example 11, wherein the atomic scale property comprises a count of relevant atoms.

Example 13: The method of example 12, wherein the relevant atoms comprise at least one of a halogen, oxygen, a three-fold coordinated carbon, or a four-fold coordinated carbon.

Example 14: The method of any of examples 11 through 13, wherein the molecular scale property comprises a count of functional groups.

Example 15: The method of example 14, wherein the count of functional groups comprises a count of at least one of an aldehyde group, an acid group, or an aromatic group.

Example 16: The method of any of examples 11 through 15, wherein the compound-scale property comprises at least one of a van der Waals surface area, a topological surface area, or a fraction of rotatable bonds.

Example 17: The method of any of examples 11 through 16, wherein the selection operator comprises a least absolute shrinkage and selection operator (LASSO).

Example 18: The method of any of examples 11 through 17, wherein the machine learning model comprises a Gaussian Process Regression (GPR) comprising a radial basis function (RBF) kernel and a five-fold cross-validation.

Example 19: The method of any of examples 11 through 18, wherein the machine learning model is trained with a training dataset comprising a plurality polymers, wherein the plurality polymers comprises a plurality of polymer classes comprising at least one of a polyoxide, a polyvinyl, a polyolefin, a polyamide, or a polyether, wherein the training dataset further comprises a plurality of chemical moieties, wherein the plurality of chemical moieties includes at least one of hydrogen, carbon, nitrogen, oxygen, sulfur, silicon, fluorine, chlorine, or bromine, wherein the training dataset further comprises at least one experimentally determined surface energy of at least one of the plurality of polymers.

Example 20: A computer readable medium includes instructions that when executed cause one or more processors to: determine a plurality of molecular descriptors, each molecular descriptor of the plurality of molecular descriptors associated with at least one of an atomic scale property, a molecular scale property, and a compound-scale property: select, by a selection operator and based on a minimization of an error, a subset of the plurality of molecular descriptors that affect a polymer surface energy: predict, via a trained machine learning model, a surface energy of an input polymer; and output the predicted surface energy.

Various examples have been described. These and other examples are within the scope of the following claims.

What is claimed is:

1. A method comprising:
determining a plurality of molecular descriptors, each molecular descriptor of the plurality of molecular descriptors associated with at least one of an atomic scale property, a molecular scale property, and a compound-scale property;
selecting, by a selection operator and based on a minimization of an error, a subset of the plurality of molecular descriptors that affect a polymer surface energy;
training a machine learning model to predict a polymer surface energy of a given polymer based on the subset of molecular descriptors; and
predicting, via the trained machine learning model, a surface energy of an input polymer.

2. The method of claim 1, wherein the polymer is a homopolymer.

3. The method of claim 1, wherein the atomic scale property comprises a count of relevant atoms.

4. The method of claim 3, wherein the relevant atoms comprise at least one of a halogen, oxygen, a three-fold coordinated carbon, or a four-fold coordinated carbon.

5. The method of claim 1, wherein the molecular scale property comprises a count of functional groups.

6. The method of claim 5, wherein the count of functional groups comprises a count of at least one of an aldehyde group, an acid group, or an aromatic group.

7. The method of claim 1, wherein the compound scale property comprises at least one of a van der Waals surface area, a topological surface area, or a fraction of rotatable bonds.

8. The method of claim 1, wherein the selection operator comprises a least absolute shrinkage and selection operator (LASSO).

9. The method of claim 1, wherein the machine learning model comprises a Gaussian Process Regression (GPR) comprising a radial basis function (RBF) kernel.

10. The method of claim 9, wherein the GPR further comprises a five-fold cross-validation.

11. A method comprising:
determining a plurality of molecular descriptors, each molecular descriptor of the plurality of molecular descriptors associated with at least one of an atomic scale property, a molecular scale property, and a compound-scale property:
selecting, by a selection operator and based on a minimization of an error, a subset of the plurality of molecular descriptors that affect a polymer surface energy;
predicting, via a trained machine learning model, a surface energy of an input polymer.

12. The method of claim 11, wherein the atomic scale property comprises a count of relevant atoms.

13. The method of claim 12, wherein the relevant atoms comprise at least one of a halogen, oxygen, a three-fold coordinated carbon, or a four-fold coordinated carbon.

14. The method of claim 11, wherein the molecular scale property comprises a count of functional groups.

15. The method of claim 14, wherein the count of functional groups comprises a count of at least one of an aldehyde group, an acid group, or an aromatic group.

16. The method of claim 11, wherein the compound-scale property comprises at least one of a van der Waals surface area, a topological surface area, or a fraction of rotatable bonds.

17. The method of claim 11, wherein the selection operator comprises a least absolute shrinkage and selection operator (LASSO).

18. The method of claim 11, wherein the machine learning model comprises a Gaussian Process Regression (GPR) comprising a radial basis function (RBF) kernel and a five-fold cross-validation.

19. The method of claim 11, wherein the machine learning model is trained with a training dataset comprising a plurality polymers, wherein the plurality polymers comprises a plurality of polymer classes comprising at least one of a polyoxide, a polyvinyl, a polyolefin, a polyamide, or a polyether, wherein the training dataset further comprises a plurality of chemical moieties, wherein the plurality of chemical moieties includes at least one of hydrogen, carbon, nitrogen, oxygen, sulfur, silicon, fluorine, chlorine, or bromine, wherein the training dataset further comprises at least one experimentally determined surface energy of at least one of the plurality of polymers.

20. A computer readable medium comprising instructions that when executed cause one or more processors to:
determine a plurality of molecular descriptors, each molecular descriptor of the plurality of molecular descriptors associated with at least one of an atomic scale property, a molecular scale property, and a compound-scale property;
select, by a selection operator and based on a minimization of an error, a subset of the plurality of molecular descriptors that affect a polymer surface energy;
predict, via a trained machine learning model, a surface energy of an input polymer, and output the predicted surface energy.
